(19) United States
(12) Patent Application Publication
(10) Pub. No.: US 2007/0294785 A1
(43) Pub. Date: Dec. 20, 2007

(54) METHODS FOR GENERATING GENETIC DIVERSITY BY PERMUTATIONAL MUTAGENESIS

(75) Inventor: Volker Heinrichs, Raleigh, NC (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Athenix Corporation, Research Triangle Park, NC (US)

(21) Appl. No.: 11/762,580

(22) Filed: Jun. 13, 2007

Related U.S. Application Data
(60) Provisional application No. 60/813,095, filed on Jun. 13, 2006.

(51) Int. Cl. A01H 1/00; C07H 21/04; C12N 9/12; C12N 9/16

(52) U.S. Cl. 800/278; 435/194; 435/196; 435/468; 435/488; 435/252.33; 536/23.2

(57) ABSTRACT

Methods for generating genetic diversity in a polynucleotide or polypeptide sequence are included. The methods include permutational mutagenesis strategies for introducing genetic diversity to alter or improve the function of the polynucleotide or polypeptide. The methods include aligning a set of homologous sequences and generating a consensus translation or a consensus sequence that encompasses the full diversity of the aligned sequences, and then incorporating that consensus translation or consensus sequence into a functional polypeptide or polynucleotide to test for altered or improved function.

### CROSS REFERENCE TO RELATED APPLICATION

This application claims the benefit of U.S. Provisional Application Ser. No. 60/813,095, filed Jun. 13, 2006, the contents of which are herein incorporated by reference in their entirety.

### REFERENCE TO SEQUENCE LISTING SUBMITTED ELECTRONICALLY

The official copy of the sequence listing is submitted electronically via EFS-Web as an ASCII formatted sequence listing with a file named “329208_SequenceListing.txt”, created on Jun. 8, 2007, and having a size of 78 kilobytes and is filed concurrently with the specification. The sequence listing contained in this ASCII formatted document is part of the specification and is herein incorporated by reference in its entirety.

### FIELD OF THE INVENTION

This invention relates to molecular biology, particularly to methods to generate genetic diversity in DNA regions of interest.

### BACKGROUND OF THE INVENTION

Directed evolution is a powerful technique to enhance or modify protein or DNA-based activities. Essentially, directed evolution co-opts the genetic paradigm and applies it to improvement of proteins and DNA. First, diversity is generated and then the diversity is subjected to a “selective pressure” such as a screen for improved enzyme activity. Thus, one key aspect for successful directed evolution is the generation of DNA libraries with broad diversity, with broad applicability. Many methods to generate diversity are known in the art, and summarized for example in Wong, et al (2006) Combinatorial Chemistry & High Throughput Screening 9(4): 271-288.

Current methods in widespread use for creating alternative proteins in a library format are error-prone polymerase chain reactions, oligo-directed mutagenesis, saturation mutagenesis, and DNA shuffling.

Error-prone PCR uses low-fidelity polymerization conditions to introduce a low level of point mutations randomly over a long sequence. In a mixture of fragments of unknown sequence, error-prone PCR can be used to mutagenize the mixture. The published error-prone PCR protocols suffer from a low processivity of the polymerase. Therefore, the protocol is unable to result in the random mutagenesis of an average-sized gene. This inability limits the practical application of error-prone PCR. Some computer simulations have suggested that point mutagenesis alone may often be too gradual to allow the large-scale block changes that are required for continued and dramatic sequence evolution. Further, the published error-prone PCR protocols do not allow for amplification of DNA fragments greater than 0.5 to 1.0 kb, limiting their practical application. In addition, repeated cycles of error-prone PCR can lead to an accumulation of neutral mutations with undesired results.

Another limitation of error-prone PCR is that the rate of down-mutations grows with the information content of the sequence. As the information content, library size, and mutagenesis rate increase, the balance of down-mutations to up-mutations will statistically prevent the selection of further improvements (statistical ceiling).

Saturation mutagenesis is an aspect of oligo-directed mutagenesis wherein one generates all possible codons over a given nucleotide region. Saturation mutagenesis over target regions can generate very large libraries, but many of the combinations of nucleotides generate non-functional proteins, stop codons, etc. Library diversity quickly becomes extremely large. Consequently, in order to identify the improved clones, one often must screen very large numbers of clones.

DNA shuffling, a method for in vitro recombination, was developed as a technique to generate mutant genes that would encode proteins with improved or unique functionality (Stemmer W P (1994) Proc Natl Acad Sci USA 91:10747-10751; Stemmer W P (1994) Nature 370:389-391). It consists of a three-step process that begins with the enzymatic digestion of genes, yielding smaller fragments of DNA, which are then allowed to randomly hybridize and are filled in to create longer fragments. Ultimately, any full-length, recombined genes that are recreated are amplified via the polymerase chain reaction. If a series of alleles or mutated genes is used as a starting point for DNA shuffling, the result is a library of recombined genes that can be translated into novel proteins, which can in turn be screened for novel functions. Genes with beneficial mutations can be shuffled further, both to bring together these independent, beneficial mutations in a single gene and to eliminate any deleterious mutations. However, if mutant alleles are neutral or interfere with each other, then there will be no genetic benefit to recombination.

Additionally, these methods can be complicated and labor intensive. In the well-established protocol of Stemmer, DNase is used to fragment DNA requiring careful optimization of the digest conditions, e.g. time, temperature, amount of nuclease and DNA (Stemmer, 1994, Nature, supra; Neylon (2004) Nucleic Acids Res. 32:1448-1459). Other methods such as the staggered extension process (Zhao et al. (1998) Nat. Biotechnol. 16:258-261) and random-priming (Shao et al. (1998) Nucleic Acids Res. 26:681-683) are limited by the DNA composition, and matters are complicated further by the lack of controllability of the range of fragment sizes generated. Methods such as RACHITT (Coco et al. (2001) Nat. Biotechnol. 19:354-359) also require DNase digests and are even more labor intensive.

Therefore, additional methods for creating polypeptides with a desired activity are needed. Accordingly, it would be advantageous to develop a method which allows for the production of large libraries of mutant polypeptides and nucleotides and the efficient selection of particular mutants for a desired activity.

### SUMMARY OF INVENTION

Methods to generate improved proteins and nucleotides are provided. The methods comprise generating polynucleotides and polypeptides with desired activities. The methods involve aligning nucleotide or amino acid sequences having regions of sequence homology and identifying regions of sequence heterogeneity. The heterologous regions are analyzed and a consensus translation (in the case of amino acid sequences) or a consensus sequence (in the case of polynucleotide sequences) is derived. A population of polynucleotides is then generated wherein the population of polynucleotides contains the consensus sequence, or encodes a population of polypeptides representing the consensus translation. Such polynucleotides would further include sufficient sequences flanking the consensus translation so that a functional sequence is generated. By “functional sequence” is intended a polypeptide or polynucleotide sequence that performs the function of at least one of the polypeptides or polynucleotides in the alignment (also referred to as a “parent sequence”). In some embodiments, this function is altered or improved in the sequence generated using the methods of the invention when compared to the function or activity of the parent sequence, thus generating a sequence with the desired characteristic or biological activity.

In some embodiments, the consensus sequences or a portion thereof is introduced into the parent sequence, replacing the corresponding region in the parent sequence. The resulting sequence is then tested for the desired biological activity or function. In accomplishing these and other objects, there has been provided, in accordance with one aspect of the invention, a method for introducing polynucleotides into a suitable host cell and growing the host cell under conditions that produce the improved polypeptide.

### DESCRIPTION OF FIGURES

FIG. 1 illustrates the design of the permutational mutagenesis library for the Q-loop region of syngrg1-SB (corresponding to positions 260 through 297 of SEQ ID NO:4). syngrg1-SB was aligned with the nucleotide sequence in the Q-loop region of grg20 (SEQ ID NO:25) and grg21 (SEQ ID NO:26). The consensus translation and oligonucleotide design are shown at the bottom of FIG. 1 and in SEQ ID NO:7 (consensus translation) and SEQ ID NO:15 (oligonucleotide design).

FIG. 2 shows an alignment of the amino acid sequences in the Q-loop core region of the glyphosate resistant clones (EVO1(2-5) (SEQ ID NO:16), L2-2 (SEQ ID NO:17), L2-3 (SEQ ID NO:18), L2-4 (SEQ ID NO:19), L2-6 (SEQ ID NO:20), L2-7 (SEQ ID NO:21), L2-8 (SEQ ID NO:22), L2-9 (SEQ ID NO:23), and L2-A (SEQ ID NO:24)). The bracket outlines the Q-loop core region. Grey shading designates positions where no alterations are observed. Positions with alterations are shown with no shading. Also included is the wild-type GRG1 amino acid sequence in this region (corresponding to amino acid positions 82 through 104 of SEQ ID NO:2).

### DETAILED DESCRIPTION OF THE INVENTION

I. Methods

The present invention is directed to a method for generating a polynucleotide sequence or population of polynucleotide sequences possessing a desired phenotypic characteristic or biological activity (e.g., altered or improved promoter function; altered or improved binding, etc.) or polynucleotide sequences encoding polypeptides with a desired phenotypic characteristic or biological activity (e.g., improved enzymatic activity, such as Vmax; higher affinity for one or more of its substrates (e.g. Km); improved resistance to enzyme inhibitors, such as competitive inhibitors, non-competitive inhibitors, and other allosteric effectors (e.g. Ki), etc). In one aspect of this invention the improved property is resistance to an herbicidal compound, including for example N-phoshonomethyl glycine (“glyphosate”). One method of identifying polypeptides that possess a desired structure or functional property (e.g., herbicide resistance) involves the screening of a large library of mutant polypeptides for individual library members which possess the desired structure or functional property conferred by the amino acid sequence of the polypeptide. The population of mutant polynucleotides comprises a subpopulation of polynucleotides that encode polypeptides which possess desired or advantageous characteristics and which can be selected by a suitable selection or screening method. The present method provides an efficient method for generating mutant or variant sequences with desired characteristics.

Library Construction Identification of a Region of Interest

In the present invention, libraries of mutated genes are generated by mutating at least one codon in a region of interest. A “region of interest” may include, for example, a region that encodes a portion of the protein that is known or suspected to be involved in its function. In the case of an enzyme, these regions can include regions important for substrate recognition, binding, or catalysis (e.g., the “active site”), or a region that is known or suspected to contribute to physical and/or chemical properties of the enzyme (e.g., solubility, shape, localization, abundance, etc.). In the case of a binding protein such as a transcription factor, the region of interest may be, for example, the DNA recognition motif, or alternatively the protein interaction motif. It is recognized that additional regions of interest can be targeted such that one or more alterations in these regions may affect the activity or function of the resulting protein or enzyme.

The method used to determine a target region for mutagenesis is not critical to the methods of the present invention. Many methods are available in the art by which one can recognize key areas of a polynucleotide or polypeptide in which to target for the methods of the inventions. The choice of the appropriate method is dependent upon the properties of the particular protein, and to some degree the preference of the practitioner.

The regions of interest may be determined by random mutagenesis techniques. For example, one may use linker scanning mutagenesis (McKnight and Kingsbury (1982) Science 217:316-324) or alanine scanning mutagenesis (Lefevre et al. (1997) Nucleic Acids Research 25(2):447-448) to identify key regions of a protein that are sensitive to such approaches. Alternatively, one may analyze the three dimensional structure of a protein, or a class of related proteins, and determine areas likely to be important for the desired property (such as substrate binding). In another embodiment, data from binding or suicide inhibitor studies may be utilized to identify key areas of the protein that are good candidates for the methods of the invention.

Regions of interest may also be identified by aligning homologous nucleotide or amino acid sequences to select conserved regions of sequence identity and regions of sequence heterogeneity (or “diversity”). For the purposes of the present invention, “homologous sequences” are sequences that share a reasonable degree of sequence similarity (e.g., greater than 50% sequence identity, greater than 55%, greater than 60%, 65%, 70%, 75%, 80%, 85%, or greater than 90%) across the entire sequence or a defined region of the sequence (for example, a binding domain or active site region). Homologous sequences can be obtained from any of the publicly available or proprietary nucleic acid databases. Public database/search services include GENEBANK®, ENTREZ®, EMBL, DDBJ and those provided by the NCBI. Many additional sequence databases are available on the internet or on a contract basis from a variety of companies specializing in genomic information generation and/or storage. A “region of sequence heterogeneity” would be one in which, for at least one position in an alignment of sequences of interest, more than one nucleotide or amino acid residue would be present across the sequences in the alignment at that position. Such a region is also referred to herein as a region of sequence diversity.

In one embodiment, one may align several related proteins of various levels of function, and from this alignment infer a region of interest. For example, this may be a particular region of amino acids that is well conserved among a class of proteins but shows an alternate amino acid pattern among a subclass of proteins of interest. For example, one may identify conserved regions among a population of EPSP synthase sequences known to be sensitive to inhibition by glyphosate herbicide and then align a subset (or subclass) of EPSP synthase sequences known to be resistant or tolerant to inhibition by glyphosate herbicide. This alignment can be used to look for deviations among the resistant EPSP synthase sequences compared to the conserved residues originally identified in the sensitive EPSP synthase sequences. Amino acid or nucleotide residues that deviate from the conserved residues in a region of interest are considered “target residues.” It is not necessary to target every residue that deviates from the conserved sequence in a region of interest. In some embodiments, it may be desirable to only target those variant residues that are known or suspected to be involved in the function or activity of the polypeptide or polynucleotide of interest (e.g., binding site or active site). In one embodiment, the target residues correspond to the amino acid positions from about 84 through about 99 of SEQ ID NO:2.

While the above section provides a detailed description of methods to determine a region of interest, other methods are known in the art. For example, regions of interest may have been described previously in the art. The method for the selection of a region of interest is not a limitation of this invention.

Library Construction Generation of a Consensus Translation

After identifying a region of interest, a consensus translation (in the case of an amino acid sequence alignment) or a consensus sequence (in the case of a nucleotide sequence alignment) is generated for this region. For the purposes of the present invention, a “consensus translation” is a compilation of amino acid sequences that represents the total amino acid diversity present in the alignment over the region of interest, and a “consensus sequence” is nucleotide sequence that represents the total nucleotide diversity in the region of interest. Where the region of interest has multiple members, one can utilize an alignment to generate the consensus translation (or consensus sequence). For example, if an alignment of multiple polypeptide sequences reveals that position 1 of the region of interest is alanine in all sequences; position 2 is arginine in one or more sequences, cysteine in one or more sequences, and trytophan in one or more sequences; and position 3 is glycine in one or more sequences and valine in all other sequences, the consensus translation for this hypothetical population of polypeptides is A-X₁-X₂ (SEQ ID NO:8), where X₁ is arginine, cysteine or tryptophan and X₂ is glycine or valine. Such a translation is said to represent the “diversity” of the region of interest in that each amino acid variation among the population of aligned polypeptides is represented in the consensus translation. Similarly, a consensus nucleotide sequence would include a nucleotide sequence that represents the nucleotide diversity present at each position in the alignment of homologous nucleotide sequences.

Methods to align polypeptide and polynucleotide sequences are well known in the art. For example, to obtain gapped alignments for comparison purposes, Gapped BLAST can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., BLASTX and BLASTN) can be used. See www.ncbi.nlm.nih.gov. Another non-limiting example of a mathematical algorithm utilized for the comparison of sequences is the ClustalW algorithm (Higgins et al. (1994) Nucleic Acids Res. 22:4673-4680). ClustalW compares sequences and aligns the entirety of the amino acid or DNA sequence, and thus can provide data about the sequence conservation of the entire amino acid sequence. The ClustalW algorithm is used in several commercially available DNA/amino acid analysis software packages, such as the ALIGNX module of the Vector NTI Program Suite (Invitrogen Corporation, Carlsbad, Calif.). After alignment of amino acid sequences with ClustalW, regions of sequence conservation and regions of sequence diversity can be identified. A non-limiting example of a software program useful for analysis of ClustalW alignments is GENEDOC™. GENEDOC™ (Karl Nicholas) allows assessment of amino acid (or DNA) similarity and identity between multiple proteins. Another non-limiting example of a mathematical algorithm utilized for the comparison of sequences is the algorithm of Myers and Miller (1988) CABIOS 4:11-17. Such an algorithm is incorporated into the ALIGN program (version 2.0), which is part of the GCG sequence alignment software package (available from Accelrys, Inc., 9865 Scranton Rd., San Diego, Calif., USA). When utilizing the ALIGN program for comparing amino acid sequences, a PAM 120 weight residue table, a gap length penalty of 12, and a gap penalty of 4 can be used.

Unless otherwise stated, GAP Version 10, which uses the algorithm of Needleman and Wunsch (1970) J. Mol. Biol. 48(3):443-453, will be used to determine sequence identity or similarity using the following parameters: % identity and % similarity for a nucleotide sequence using GAP Weight of 50 and Length Weight of 3, and the nwsgapdna.cmp scoring matrix; % identity or % similarity for an amino acid sequence using GAP weight of 8 and length weight of 2, and the BLOSUM62 scoring program. Equivalent programs may also be used. By “equivalent program” is intended any sequence comparison program that, for any two sequences in question, generates an alignment having identical nucleotide residue matches and an identical percent sequence identity when compared to the corresponding alignment generated by GAP Version 10.

Library Construction Design of DNA Oligonucleotides

After generating a consensus translation, oligonucleotides are designed to generate a library representing polynucleotides encoding the diversity of the consensus translation. For example, in the case of the hypothetical region of interest described above, a set of oligonucleotides representing the diversity of the consensus translation would include at least one oligonucleotide that encodes of each of the following amino acid sequences (single letter amino acid code): ARG, ARV, ACG, ACV, AWG and AWV (SEQ ID NO:9-14, respectively).

In one aspect, the invention comprises synthesizing one or more oligonucleotides corresponding to at least one region of sequence diversity. An “oligonucleotide” (or “oligo”) refers to either a single stranded polydeoxynucleotide or two complementary polydeoxynucleotide strands which may be chemically synthesized. Such synthetic oligonucleotides may or may not have a 5′ phosphate. Typically sets of oligonucleotides are produced, e.g., by sequential or parallel oligonucleotide synthesis protocols.

In one embodiment, the population (or “set”) of oligonucleotides encoding the target protein's region of interest is degenerate at each codon to the extent that the population of oligos encodes the full diversity of the consensus translation, while minimizing “additional diversity” (described infra). Previous methods have utilized oligos with fully randomized codons at each of the target residues in the region of interest. A fully randomized codon is represented by the sequence “N,N,N” where “N” can be any one of the nucleotide bases A, T, C or G. Thus, there are sixty four possible nucleotide sequences represented by a fully randomized codon that uses A, T, G and C.

In the present invention, oligos corresponding to a region of interest are designed to be degenerate only at those target positions where a base change results in an alteration in an encoded polypeptide sequence. This has the advantage of requiring fewer degenerate oligonucleotides to achieve the same degree of diversity in encoded products, thereby simplifying the synthesis of the population of mutagenized oligonucleotides. Oligonucleotides generated by permutational methods will have substantially fewer than sixty four possible codons at each target position, thus reducing the library size while still maintaining the diversity of the consensus translation in the library.

Ideally, oligonucleotides are designed so that only encoded amino acid alterations of the consensus are created as a result of the synthesis. However, due to the degeneracy of the genetic code, and the current methods for DNA synthesis, it is more typical that some “additional diversity” is generated by the synthesis strategy. For example, if one wants to create a consensus translation of aspartic acid and lysine, using the codons G/A/T for aspartic acid and A/A/G for lysine generates the consensus codon R(A or G)/A/K(T or G). Thus, an oligonucleotide encompassing this diversity will have the desired codons G/A/T (encoding aspartic acid), A/A/G (encoding lysine) but will also have G/A/G (encoding glutamic acid), and A/A/T encoding (asparagine). The design of the oligonucleotides should be such to minimize this additional diversity. One method for minimizing this diversity is to select among all possible codons capable of representing each member of the consensus translation for those codons (the “preferred codons”) that generate the minimal amount of additional diversity. One then designs the oligonucleotides to generate these preferred codons for each position of the consensus translation to the extent possible. For example, if the consensus translation has an isoleucine and a threonine at a target position, the use of the codon A/T/T for isoleucine in combination with A/C/T for threonine generates the consensus codon A/(T or C)/T. This consensus codon will only encode isoleucine and threonine. However, the use of codon A/T/T for isoleucine in combination with A/C/G for threonine will result in the consensus codon A/(T or C)/(T or G). This consensus codon encodes isoleucine, threonine and methionine (with “methionine” in this example representing the “additional diversity”).

In a further embodiment, the oligonucleotides are designed such that the degeneracy is spread among more than one oligonucleotide, yet nonetheless generates a library that comprises the full diversity of the consensus translation. In a preferred aspect of this invention, the number of amino acids in a consensus translation is partitioned between two or more populations of oligonucleotides. The best method to perform this partitioning is to first select the target position of the consensus translation that has the highest diversity (e.g., the highest number of amino acid variations at this position). Then, for this position, the total number of amino acids to be encoded is partitioned into two or more populations of oligonucleotides such that one population of oligonucleotides will encode one amino acid at a given target position in the consensus translation, and a second population of oligonucleotides will encode a different amino acid at that same target position, etc. The result is that the degeneracy in each population of oligonucleotides is greatly reduced, yet the library still achieves the full diversity of the consensus translation.

In another aspect of this invention, this approach is applied to more than one target position in the region of interest. This results in further reduction in undesired (“additional”) diversity, while maintaining the diversity of the consensus translation. Usually a practical limit occurs due to the increasing number of oligonucleotides required to utilize this preferred approach. For example, to utilize this approach for two target positions, each with six amino acids in the consensus translation, requires the synthesis of 36 populations of oligonucleotides instead of a single population of oligonucleotides that encodes each of the six amino acids at each of the two target positions. In this method, the degeneracy of the library is greatly reduced (i.e., minimization of the “additional diversity” described above), while still capturing the full diversity of the consensus translation. Ultimately, it is desired to utilize this design strategy to include every amino acid of the region of interest, unless the number of oligonucleotides becomes excessive (determined largely by the resources available to the practitioner).

Developments in DNA chemistry have lead to the discovery of quite a large number of variable (non-natural) nucleotides, such as 7-deazoguanosine, inosine, and the like. These nucleotides often have broader hydrogen bonding preferences than natural nucleotides, and can be useful to help reduce the number of oligonucleotides required.

In a further embodiment of the invention, the mutant oligonucleotides are typically designed to incorporate restriction sites to facilitate cloning and expression of the mutated gene sequences. The restriction sites may occur naturally in the parent nucleotide sequence, or may be inserted into the sequence, for example, using site-directed mutagenesis. Insertion of a restriction site should be done in a manner that does not disrupt the activity or function of the polynucleotide or the encoded polypeptide. Sequences that are cleaved by restriction endonucleases (“restriction sites”) are well known in the art.

Oligonucleotides are typically synthesized chemically according to the solid phase phosphoramidite triester method described by Beaucage and Caruthers (1981), Tetrahedron Letts. 22(20):1859-1862, for example, using an automated synthesizer, as described in Needham-VanDevanter et al. (1984) Nucleic Acids Res. 12:6159-6168. A wide variety of equipment is commercially available for automated oligonucleotide synthesis. Multi-nucleotide synthesis approaches (e.g., tri-nucleotide synthesis), as discussed supra are also useful.

Library Construction Annealing of Oligonucleotides and Cloning of Libraries

After designing and synthesizing the population(s) of oligonucleotides, the oligonucleotides are introduced into the polynucleotide of interest to generate a polynucleotide with desired characteristics, or a polynucleotide that encodes a polypeptide with desired characteristics. In this context, “introduced” means to insert the sequences of the oligonucleotides into the polynucleotide of interest such that the sequence in the region of interest is replaced by the oligonucleotide sequence.

In one embodiment, the population of oligonucleotides is introduced into the polynucleotide of interest by annealing the oligonucleotides and then ligating the population of oligonucleotides into a vector comprising the polynucleotide of interest to generate a DNA library. This can be accomplished, for example, by identifying or introducing (for example, by site-directed mutagenesis) unique restriction sites into the sequences flanking the target region in the polynucleotide of interest, and designing the oligonucleotide(s) to contain the same unique restriction sites. In this example, the target region may be easily replaced by enzymatic digestion with the restriction endonuclease enzyme(s) that will specifically cleave the polynucleotide within the unique restriction site(s) in both the target region of the polynucleotide of interest and in the oligonucleotide(s). The digested oligonucleotides are then ligated (e.g., introduced) into the digested vector comprising the polynucleotide of interest using standard molecular biology techniques. The oligonucleotides may be ligated without the need for extension (e.g., polymerase-based chain extension). The resulting library is transformed into a host cell and methods for assaying function or activity are then utilized to identify polynucleotides or polypeptides having the desired biological activity (e.g., desired characteristic).

In another embodiment, the oligonucleotides can be introduced into the polynucleotide of interest using polymerase chain reaction, wherein the oligonucleotides corresponding to the region(s) of sequence heterogeneity are annealed to the polynucleotide of interest and the variant polynucleotides are generated by primer extension using a thermostable DNA polymerase and further techniques well known to those of skill in the art.

In another embodiment, polynucleotides containing the consensus translation are synthesized de novo. These polynucleotides would include the consensus domain (or consensus sequence) as well as sequences flanking the consensus translation (or consensus sequence) sufficient to result in a functional sequence (e.g., a functional polypeptide such as an enzyme, a receptor, a binding protein, etc, or a functional polynucleotide such as a promoter).

Expression of the Library of Variants in Cells

The variant polynucleotides with increased diversity (or those polynucleotides encoding polypeptides with increased diversity) are typically expressed in a host cell to obtain the desired phenotypic characteristic or biological activity (e.g., expression (and/or secretion) of a protein, resistance to a drug or infective agent, etc). The “variant polynucleotides” are those that are generated using the methods described supra. The host cell could be any cell, including (but not limited to) bacterial cells, such as E. coli or Bacillus; cultured eukaryotic cells, such as a HU293 cell; or plant cells. Host cells containing the variant polynucleotides of interest can be cultured in conventional nutrient media modified as appropriate for activating promoters, selecting transformants or amplifying genes. In the case of cultured cells, the culture conditions, such as temperature, pH and the like, are those previously used with the host cell selected for expression, and will be apparent to the skilled artisan.

Plant Transformation

The polynucleotides identified by the methods of the present invention can be introduced into a plant or plant cell such that expression of the polynucleotide confers an improved property upon the plant or plant cell. By “introduced” or “introducing” in this context is intended to present to the plant the polynucleotide in such a manner that the polynucleotide gains access to the interior of a cell of the plant. The methods of the invention do not require that a particular method for introducing a polynucleotide into a plant be used, only that the polynucleotide gains access to the interior of at least one cell of the plant.

Introduction of a polynucleotide into plant cells is accomplished by one of several techniques known in the art, including but not limited to electroporation or chemical transformation (See, for example, Ausubel, ed. (1994) Current Protocols in Molecular Biology (John Wiley and Sons, Inc., Indianapolis, Ind.). Markers conferring resistance to toxic substances are useful in identifying transformed cells (having taken up and expressed the test polynucleotide sequence) from non-transformed cells (those not containing or not expressing the test polynucleotide sequence). In one aspect of the invention, genes expressing variants generated by the methods of the invention may be screened to identify variants conferring improved properties, such as the ability to act as a marker to assess introduction of DNA into plant cells. Similarly, the improved protein identified by the methods of the invention, may be useful as a marker to assess introduction of DNA into plant cells. “Transgenic plants” or “transformed plants” or “stably transformed” plants, cells, tissues or seed refer to plants that have incorporated or integrated exogenous polynucleotides into the plant cell. By “stable transformation” is intended that the polynucleotide construct introduced into a plant integrates into the genome of the plant and is capable of being inherited by progeny thereof.

Screening

Methods for screening for altered or improved activity or function of a polynucleotide or polypeptide of interest are typically well known to those of skill in the art to which the polynucleotide or polypeptide of interest pertains. The motivation to alter or improve a polynucleotide or polypeptide of interest is often triggered or supported by knowledge of the polynucleotide's or polypeptide's function or activity. As such, methods to screen for activity or function of the polynucleotides or polypeptides generated using the methods of the invention are well known or can be derived without undue experimentation by one of skill in the relevant art.

The clones which exhibit improved properties (such as for example, improved catalytic activity on substrate (V and/or Km), improved binding affinity, reduced product inhibition, ability to tolerate altered reaction conditions such as pH, temperature, salt, or organic solvents, or improved tolerance of inhibitors, improved resistance to inhibition by herbicide) may then be sequenced to identify the polynucleotide sequence encoding the polypeptide having the enhanced activity (e.g., herbicide resistance). Methods for isolating and identifying sequences from “improved” clones are well known in the art and are described elsewhere herein (e.g., Brakmann (2001) ChemBiochem 2: 865-871).

Further Aspects of the Invention

Use of the methods of the invention followed by screening will often lead to (1) isolation of clones with altered or improved function or (2) generation of large amounts of data regarding the effects of mutations upon the residues at each position of the region of interest. For example, this data may be collected by (a) generating a library for a region of interest (2) screening the library as expressed in host cells, and identifying a number of clones that retain activity (for example, at approximately the wild-type level) (c) determining the DNA sequence (and the corresponding amino acid sequence) of the region of interest for the large number of clones so isolated.

The resulting data about (1) positions that cannot be changed, (2) those that can be freely altered in survivors, and (3) those that can tolerate limited alteration that results from use of this invention is very valuable.

The information resulting from use of the methods of the invention allows one to target a smaller subset of positions for further mutagenesis, either by a permutational approach that is restricted to fewer positions (by, for example, incorporating a larger amount of diversity in these positions by including additional proteins into the alignments or by choosing to incorporate conserved amino acids, etc.), or alternatively by saturation mutagenesis or other mutagenesis strategies. The choice of mutagenesis method depends on the number of positions that are mutable. For instance, saturation mutagenesis may be preferred in the case that there are a small number (2-6 amino acids) that are mutable. However, permutational mutagenesis is optimal when there are a large number of sequences that may be aligned to generate a region of interest or where the number of mutable residues is greater than about 6 residues.

The following examples are offered by way of illustration and not by way of limitation.

### EXPERIMENTAL

### Example 1

### Permutational Mutagenesis of syngrg1-SB

### syngrg1 Design and Expression

A novel gene sequence encoding the GRG1 protein (SEQ ID NO:1 and 2; U.S. patent application Ser. No. 10/739,610 filed Dec. 18, 2003) was designed and synthesized. This sequence is provided as SEQ ID NO:3 (and in U.S. patent application Ser. No. ______ entitled “Improved EPSP Synthases: Compositions and Methods of Use” and filed concurrently herewith, which is herein incorporated by reference in its entirety). This open reading frame, designated “syngrg1” herein, was cloned into the expression vector pRSF1b (Invitrogen) by methods known in the art

Site-Directed Mutagenesis of GRG1

U.S. patent application Ser. No. 11/651,752, filed Jan. 10, 2007 (herein incorporated by reference) discloses the Q-loop as an important region in conferring glyphosate resistance to EPSP synthases. The region of the Q-loop can be identified by aligning amino acid sequences with the conserved arginine in the amino acid region corresponding to positions 80-105 of SEQ ID NO:2. It is recognized that the amino acid number may vary by about plus or minus 20, 15, 10, 9, 8, 7, 6, 5, 4, 3, 2 or 1 amino acid(s) on either side of the Q-loop. For the purposes of the present invention, discussion of the Q-loop will be further restricted to a region comprising the “core” region of the Q-loop spanning from the isoleucine corresponding to amino acid position 84 of SEQ ID NO:2 to the isoleucine corresponding to amino acid position 99 of SEQ ID NO:2.

Herein a position number is assigned to the amino acids in this core region to simplify referral to each amino acid residue in this region. Thus, the positions of the Q-loop core correspond to amino acids 84 through 99 of SEQ ID NO:2 (I-D-C-G-E-S-G-L-S-I-R-M-F-T-P-I) and are herein designated as follows:

TABLE 1
Designation of Position Coordinates for Q-loop Core amino acids

| Amino Acid in | |
|---|---|
| GRG1 (SEQ ID | |
| NO: 2 | Designated Position |
| (single letter code) | in Q-loop Core |
| I | Position 1 |
| D | Position 2 |
| C | Position 3 |
| G | Position 4 |
| E | Position 5 |
| S | Position 6 |
| G | Position 7 |
| L | Position 8 |
| S | Position 9 |
| I | Position 10 |
| R | Position 11 |
| M | Position 12 |
| F | Position 13 |
| T | Position 14 |
| P | Position 15 |
| I | Position 16 |

A variant of syngrg1, referred to herein as syngrg1-SB (SEQ ID NO:4) (see U.S. patent application Ser. No. ______, entitled “Improved EPSP Synthases: Compositions and Methods of Use, filed concurrently herewith and incorporated by reference in its entirety), was generated using site-directed mutagenesis to create convenient Spe I and BstB I restriction sites flanking the Q-loop.

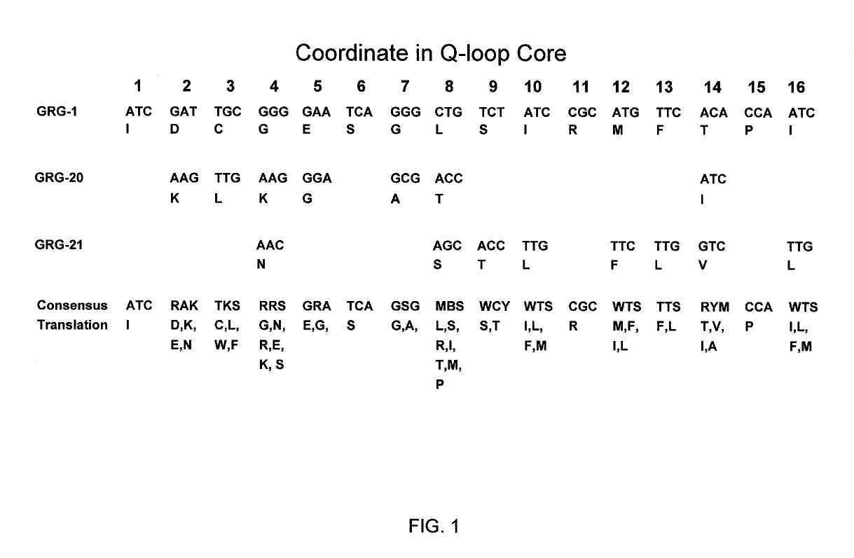

The amino acid sequences of GRG1, GRG20 (SEQ ID NO:5) (see U.S. patent application Ser. No. 11/651,752, filed Jan. 10, 2007) and GRG21 (SEQ ID NO:6) (see U.S. patent application Ser. No. 11/651,752) were aligned and a consensus translation of amino acids developed (FIG. 1, SEQ ID NO:7).

A series of oligonucleotides (represented by the consensus sequence of SEQ ID NO:15) was designed to introduce the diversity represented in FIG. 1, which covers the full diversity of the consensus translation of the Q-loop core as shown in Table 1. Positions 1, 6, 11, and 15 are absolutely conserved between GRG1, GRG20, and GRG21. The potential diversity generated by this approach is shown as the consensus translation in FIG. 1 and in SEQ ID NO:7.

Oligonucleotides were resuspended in 10 mM Tris-HCl pH 8.5 at a concentration of 10 μM. To form double stranded DNA molecules, complementary oligonucleotides were mixed and incubated as follows: 95° C. for 1 minute; 80° C. for 1 minute; 70° C. for 1 minute; 60° C. for 1 minute; and 50° C. for 1 minute. The annealed oligonucleotides were ligated to pRSF1b-syngrg1-SB digested with Spe I and BstB I, and treated with calf alkaline phosphatase. Test ligations were transformed into BL21*DE3 (Invitrogen) and plated on LB-kanamycin. From these test transformations, the library was estimated to contain approximately 180,000 clones. Twenty clones were randomly selected from the clones growing on LB and sequenced. Nineteen of the 20 clones were found to encode full length, in-frame proteins in the Q-loop region, despite the generation of a large amount of diversity in the region. High degrees of variation were seen (at all 13 target positions) in the twenty clones sequenced, suggesting that the library diversity approached its theoretical level (data not shown).

Screening for Glyphosate Resistance on Plates

Library ligations were transformed into BL21*DE3 competent E. coli cells (Invitrogen). The transformations were performed according to the manufacturer's instructions with the following modifications. After incubation for 1 hour at 37° C. in SOC medium, the cells were sedimented by centrifugation (5 minutes, 1000×g, 4° C.). The cells were washed with 1 ml M63+, centrifuged again, and the supernatant decanted. The cells were washed a second time with 1 ml M63+ and resuspended in 200 ul M63+.

For selection of mutant GRG1 enzymes conferring glyphosate resistance to E. coli, the cells were plated onto M63+ agar medium plates containing 50 mM glyphosate, 0.05 mM IPTG (isopropyl-beta-D-thiogalactopyranoside), and 50 ug/ml kanamycin. M63+ medium contains 100 mM KH₂PO₄, 15 mM (NH₄)₂SO₄, 50 μM CaCl₂, 1 μM FeSO₄, 50 μM MgCl₂, 55 mM glucose, 25 mg/liter L-proline, 10 mg/liter thiamine HCl, sufficient NaOH to adjust the pH to 7.0, and 15 g/liter agar. The plates were incubated for 36 hours at 37° C.

Determination of Variant Residues

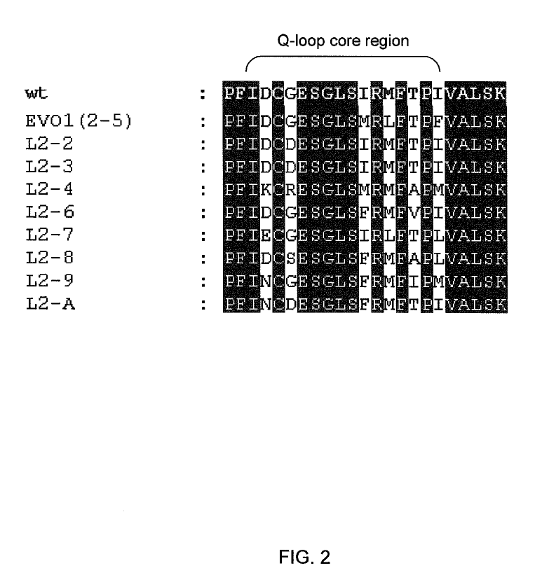

The library generated by the methods described above has a theoretic diversity of over 2,000,000 clones, and approximately 180,000 clones were tested for glyphosate resistance. Nine clones were identified by growth on 50 mM glyphosate plates (FIG. 2). DNA was isolated from these nine clones, and the DNA sequence of the Q-loop core region of each clone was determined. Comparison of the resulting DNA sequences against the DNA sequences of the randomly sampled clones (growing on LB-kanamycin) showed that many of the 13 core residues altered in this library were intolerant of variation. For example, position 8 of the core was represented by the amino acids leucine, isoleucine, serine, arginine, methionine, and proline. However, every glyphosate resistant clone (growing on 50 mM glyphosate) isolated contained a leucine at position 8. This result suggests that, under the conditions disclosed herein, substitution of the other amino acids for leucine negatively affected the enzymatic activity of the EPSP synthase, the glyphosate resistance of the resulting EPSP synthase, or both properties. Thus, this method is useful to “map” the mutable amino acids in the Q-loop core region.

### Example 2

### Permutational Mutagenesis of Genes for Insect or Nematode Control

Permutational mutagenesis is also useful for developing new insect and nematode toxin genes with altered and/or improved properties, such as effective control of a broader class of insects, or improved activity upon commercially relevant nematodes.

Permutational mutagenesis may be used to improve the activity or change the specificity of proteins that are insecticidal or nematicidal (e.g. cry proteins from Bacillus thuringiensis).

Choosing Domains for Mutagenesis

In order to choose a region of interest, one may align the amino acid sequences of, for example, known endotoxin genes, as well as utilize the knowledge in the art of regions of these endotoxin genes important for activity (e.g., regions involved in binding to insect gut receptors). A variety of endotoxin genes, as well as functional domains therein, are well known in the art (see, for example, Bravo (1997) J. Bacteriol. 179(9):2793-801; Crickmore et al. (1998) Microbiol. Molec. Biol. Rev. 62:807-813; and Crickmore et al. (2004) Bacillus thuringiensis Toxin Nomenclature on the world wide web at lifesci.sussex.ac.uk/Home/Neil_Crickmore/Bt).

Design of Oligonucleotides

The oligonucleotides are designed to capture the diversity of the consensus translation, and to minimize the unwanted diversity using methods described supra.

Screening of Mutant Libraries

A preliminary screen to eliminate mutations that insert spurious “stop” codons or destabilize the protein may be incorporated. The library should be generated in an expression vector that will insert a translational tag (e.g., a 6×His tag, a biotin binding domain, an antibiotic resistance gene, etc.) at the C terminus of the protein. The tag will be present only if the complete protein is translated in the correct reading frame. The presence of the tag may be detected by colony lifts or, in the case of the antibiotic resistance marker, by antibiotic selection. The individual colonies may then be grown in a multi-well format and screened by bioassay. Assays for measuring pesticidal activity are known in the art. In one method, the altered or improved polypeptide of the invention is mixed and used in feeding assays. See, for example Marrone et al. (1985) J. of Economic Entomology 78:290-293. Such assays can include contacting plants with one or more pests and determining the plant's ability to survive and/or cause the death of the pests. The methods of the invention can be used to evolve any pesticidal protein of interest.

Alternative methods for assessing altered or improved activity against a pest of interest are described in U.S. patent application Ser. No. 10/969,364, which is herein incorporated by reference in its entirety. This assay measures the binding activity of a protein to brush border membrane vesicles (BBMV) from target pests. Individual colonies are grown in 96 well format and the crude extracts incubated with brush border membrane vesicles prepared from the foregut of the target pest. The complex may be captured in a 96 well format in commercially available plates that are conjugated with either nickel or biotin, or an antibody specific to the protein or the tag. The BBMV binding can then be detected by measuring, for example, alkaline phosphatase activity (in the case of lepidopteran insects) or acid phosphatase activity (in the case of nematodes). Alternatively, the complex could be captured by reaction with a specific antibody, incubation with Protein A agarose, precipitated by centrifugation and analyzed using BBMVs as described above.

### Example 3

### Permutational Mutagenesis of a DNA Region for Improved Protein Binding

One may utilize the methods of the present invention to generate altered or improved DNA binding regions. The polynucleotide sequence of several DNA binding regions can be aligned with similar structures, for example, ubiquitin promoter regions. Then a region of interest can be selected (for example, an RNA polymerase binding region). From this alignment, a consensus translation that captures the diversity in this region can be derived, and oligonucleotides that recreate the diversity of the consensus translation can be synthesized and used to generate a library of such sequences in the larger context of (for example) the ubiquitin promoter. This library can be screened for function (for example, improved transcription) by methods known in the art. For example, a gene for an easily quantified protein, such as Green fluorescent protein, can be placed under the control of the ubiquitin promoter sequences generated by the methods of the present invention. The library is then introduced into cells, such as tissue culture cells, and then the cells are assayed for a desired property, for example, increased expression, or expression at a particular stage of the cell cycle.

### Example 4

### Permutational Mutagenesis to Alter Orotein Regulatory Signals

The methods of the present invention may be utilized to generate altered proteins that are still functional, but are no longer subject to protein-based post-translational regulation. For example, by this method one may develop novel yeast chitin synthetases that are insensitive to the translational regulation usually exerted upon yeast chitin synthases.

### Example 5

### Other Uses for Permutational Mutagenesis

The methods of the present invention can be used to improve virtually any polynucleotide or polypeptide sequence.

For example, the receptor binding regions of various molecules cytokines (including IFNα, IFNβ, IFNγ, G-CSF, IL-2, IL-12, and others) can be targeted for evolution in order to, for example, increase receptor affinity to increase cytokine potency. The methods could also be used to improve or change receptor recognition by these cytokines. Many human cytokines are pluripotent and act on several cell types. As a result, therapeutic cytokines often cause undesirable side effects in humans. By evolving them to recognize receptors more specifically, these side effects may be ameliorated.

In another embodiment, antibodies (for example anti-TNFalpha, anti-Her2, and others) are evolved to increase affinity, increase specificity, and/or reduce Fc receptor binding to reduce complement activation.

In another embodiment, immunostimulatory molecules (such as CTLA-4, CD40, B7, others) are evolved to increase affinity and to increase or change receptor specificity.

In another embodiment, vaccines (for example against HBV, HIV, HPV, HCV, malaria, and others) could be evolved to increase potency, affinity and to evolve cross-strain protective vaccines.

In another embodiment, regulatory RNAs (for example snRNA, RNAi, and others) are evolved using the methods of the present invention. These RNAs are involved in RNA splicing (snRNA) and RNA degradation (RNAi), usually by base pairing with short RNA sequences on their target RNAs. Permutational mutagenesis could be used to increase affinity and, importantly, to alter target specificity. Depending on the intended use of the RNA species, an increase or a decrease in the stability of the RNA molecule is altered.

The binding sites of protein factors regulating RNA splicing (for example SR proteins) or transcription can also be evolved by permutational mutagenesis to increase or alter binding specificity.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. A method of generating a polynucleotide encoding a polypeptide having a desired characteristic comprising:
a) aligning a plurality of polypeptides having regions of sequence homology to identify one or more regions of sequence heterogeneity;
b) generating a consensus translation for at least a first region of sequence heterogeneity;
c) generating a population of polynucleotides, wherein said population of polynucleotides encodes a population of polypeptides, wherein the sequence corresponding to the at least a first region of sequence heterogeneity in the population of polypeptides consists of the consensus translation generated in step (b);
d) ligating said population of polynucleotides into an expression vector construct;
e) expressing the construct generated in step (d) in a host cell to provide polypeptide expression products; and,
f) testing for said desired characteristic.

2. The method of claim 1, further comprising repeating steps (b)-(f), wherein said consensus translation is generated for a second region of heterogeneity.

3. The method of claim 1, wherein the population of polynucleotides generated in step (e) encodes functional polypeptides.

4. The method of claim 1, wherein the polypeptide having a desired characteristic is an enzyme.

5. The method of claim 1, wherein the polypeptide having a desired characteristic is a binding protein.

6. The method of claim 1, polypeptide having a desired characteristic is a structural protein.

7. The method of claim 4, wherein the enzyme is EPSP synthase.

8. The method of claim 7, wherein the EPSP synthase is encoded by a synthetic polynucleotide sequence that has been designed for expression in a plant.

9. The method of claim 7, wherein the one or more regions of sequence heterogeneity comprise at least a portion of the EPSP synthase active site.

10. The method of claim 9, wherein said one or more regions of sequence heterogeneity comprise an amino acid sequence corresponding to positions 84 through 99 of SEQ ID NO:2.

11. The method of claim 7 wherein said host cell is E. coli and the generated EPSP synthase is resistant to inhibition by glyphosate herbicide, wherein said resistance is assessed by growth of said E. coli in the presence of glyphosate.

12. A method of generating a polynucleotide having a desired characteristic comprising:
a) aligning a plurality of polynucleotides having regions of sequence homology to identify one or more regions of sequence heterogeneity;
b) generating a consensus sequence for at least a first region of sequence heterogeneity;
c) generating a population of polynucleotides, wherein the sequence corresponding to the at least a first region of sequence heterogeneity consists of the consensus sequence generated in step (b);
d) ligating said population of polynucleotides into an expression vector construct; and,
e) testing resulting polynucleotides for said desired characteristic.

13. The method of claim 12, further comprising repeating steps (b)-(e), wherein said consensus sequence is generated for a second region of heterogeneity.

14. The method of claim 12, wherein said polynucleotide of interest is a promoter of transcription.

15. The method of claim 12, wherein said polynucleotide of interest is a protein binding region.


SEQUENCE LISTING (26 sequences)

SEQ ID NO: 1 - DNA, 1398 bp, Enterobacteriaceae
  CDS (103)...(1398)
aaaaaaggaaatgaactatgtgttgctggaaaaagtagggaagggagtggtgaagagtattccactggttcaattagaaaaaatcattcaaggattaccaaagtgaaagtaacaatacagcccggagatctgactggaattatccagtcacccgcttcaaaaagttcgatgcagcgagcttgtgctgctgcactggttgcaaaaggaataagtgagatcattaatcccggtcatagcaatgatgataaagctgccagggatattgtaagccggcttggtgccaggcttgaagatcagcctgatggttctttgcagataacaagtgaaggcgtaaaacctgtcgctccttttattgactgcggtgaatctggtttaagtatccggatgtttactccgattgttgcgttgagtaaagaagaggtgacgatcaaaggatctggaagccttgttacaagaccaatggatttctttgatgaaattcttccgcatctcggtgtaaaagttaaatctaaccagggtaaattgcctctcgttatacaggggccattgaaaccagcagacgttacggttgatgggtccttaagctctcagttccttacaggtttgttgcttgcatatgcggccgcagatgcaagcgatgttgcgataaaagtaacgaatctcaaaagccgtccgtatatcgatcttacactggatgtgatgaagcggtttggtttgaagactcccgagaatcgaaactatgaagagttttatttcaaagccgggaatgtatatgatgaaacgaaaatgcaacgatacaccgtagaaggcgactggagcggtggtgcttttttactggtagcgggggctattgccgggccgatcacggtaagaggtttggatatagcttcgacgcaggctgataaagcgatcgttcaggctttgatgagtgcgaacgcaggtattgcgattgatgcaaaagagatcaaacttcatcctgctgatctcaatgcatttgaatttgatgctactgattgcccggatctttttccgccattggttgctttggcgtcttattgcaaaggagaaacaaagatcaaaggcgtaagcaggctggcgcataaagaaagtgacagaggattgacgctgcaggacgagttcgggaaaatgggtgttgaaatccaccttgagggagatctgatgcgcgtgatcggagggaaaggcgtaaaaggagctgaagttagttcaaggcacgatcatcgcattgcgatggcttgcgcggtggctgctttaaaagctgtgggtgaaacaaccatcgaacatgcagaagcggtgaataaatcctacccggatttttacagcgatcttaaacaacttggcggtgttgtatctttaaaccatcaatttaatttctcatga

SEQ ID NO: 2 - protein, 431 aa, Enterobacteriaceae
MKVTIQPGDLTGIIQSPASKSSMQRACAAALVAKGISEIINPGHSNDDKAARDIVSRLGARLEDQPDGSLQITSEGVKPVAPFIDCGESGLSIRMFTPIVALSKEEVTIKGSGSLVTRPMDFFDEILPHLGVKVKSNQGKLPLVIQGPLKPADVTVDGSLSSQFLTGLLLAYAAADASDVAIKVTNLKSRPYIDLTLDVMKRFGLKTPENRNYEEFYFKAGNVYDETKMQRYTVEGDWSGGAFLLVAGAIAGPITVRGLDIASTQADKAIVQALMSANAGIAIDAKEIKLHPADLNAFEFDATDCPDLFPPLVALASYCKGETKIKGVSRLAHKESDRGLTLQDEFGKMGVEIHLEGDLMRVIGGKGVKGAEVSSRHDHRIAMACAVAALKAVGETTIEHAEAVNKSYPDFYSDLKQLGGVVSLNHQFNFS

SEQ ID NO: 3 - DNA, 1296 bp, Artificial Sequence
  syngrg1
  CDS (1)...(1296)
atgaaggtgacaatccagcctggcgatctcacaggcatcattcagagcccagcgtcaaagtcttcaatgcagagagcgtgcgcggcggccctggtggcgaaggggatctcagaaatcatcaaccctgggcatagcaacgatgataaggccgcgagagatatcgtgagccgtcttggggccagacttgaagatcagccagatggcagcctccagatcacttcagaaggcgttaagccagtggcgcctttcatcgattgcggggaatcagggctgtctatccgcatgttcacaccaatcgtggcgctctcaaaggaagaagtgacaatcaaggggtcagggtcactcgttactcgccctatggatttcttcgatgaaatcctgccacatctgggcgtgaaggtgaagtcaaatcaggggaagctccctctggttatccaggggccacttaagccagcggatgttacagttgatgggtctctctcatctcagttcctgacaggcctcctgcttgcctacgccgcggcggatgccagcgatgttgccatcaaggtgactaacctgaagtcacgtccttacatcgatcttactcttgatgttatgaagcgtttcggcctcaagactcctgaaaaccgcaactacgaagagttctacttcaaggccgggaacgtgtacgacgaaacaaagatgcagcgttacactgttgaaggggattggtcagggggcgcgttcctgctcgttgcgggggccatcgccgggccaatcactgttcgtggccttgatatcgcgtcaactcaggcggataaggcgatcgttcaggcgctcatgagcgccaacgccgggatcgcgatcgatgccaaggaaatcaagctgcatcctgccgatctgaacgccttcgagttcgatgccactgattgccctgatctcttcccaccactcgtggccctcgcctcatactgcaagggggaaacaaagatcaagggcgtgagccgccttgcgcataaggaatctgatagagggctgactcttcaggatgagttcgggaagatgggcgttgaaatccatcttgaaggggatctcatgcgtgtgatcggcgggaagggggtgaagggcgccgaagttagctcacgtcatgatcatcgcatcgccatggcgtgcgccgtggcggcgctcaaggccgttggggaaacaacaatcgaacatgccgaagcggttaacaagtcttaccctgatttctactcagatttgaagcagctcgggggcgtggtgtctctgaaccatcagttcaacttctcttag

SEQ ID NO: 4 - DNA, 1296 bp, Artificial Sequence
  syngrg1-SB
  CDS (1)...(1296)
atgaaggtgacaatccagcctggcgatctcacaggcatcattcagagcccagcgtcaaagtcttcaatgcagagagcgtgcgcggcggccctggtggcgaaggggatctcagaaatcatcaaccctgggcatagcaacgatgataaggccgcgagagatatcgtgagccgtcttggggccagacttgaagatcagccagatggcagcctccagatcactagtgaaggcgttaagccagtggcgcctttcatcgattgcggggaatcagggctgtctatccgcatgttcacaccaatcgtggcgctttcgaaggaagaagtgacaatcaaggggtcagggtcactcgttactcgccctatggatttcttcgatgaaatcctgccacatctgggcgtgaaggtgaagtcaaatcaggggaagctccctctggttatccaggggccacttaagccagcggatgttacagttgatgggtctctctcatctcagttcctgacaggcctcctgcttgcctacgccgcggcggatgccagcgatgttgccatcaaggtgactaacctgaagtcacgtccttacatcgatcttactcttgatgttatgaagcgtttcggcctcaagactcctgaaaaccgcaactacgaagagttctacttcaaggccgggaacgtgtacgacgaaacaaagatgcagcgttacactgttgaaggggattggtcagggggcgcgttcctgctcgttgcgggggccatcgccgggccaatcactgttcgtggccttgatatcgcgtcaactcaggcggataaggcgatcgttcaggcgctcatgagcgccaacgccgggatcgcgatcgatgccaaggaaatcaagctgcatcctgccgatctgaacgccttcgagttcgatgccactgattgccctgatctcttcccaccactcgtggccctcgcctcatactgcaagggggaaacaaagatcaagggcgtgagccgccttgcgcataaggaatctgatagagggctgactcttcaggatgagttcgggaagatgggcgttgaaatccatcttgaaggggatctcatgcgtgtgatcggcgggaagggggtgaagggcgccgaagttagctcacgtcatgatcatcgcatcgccatggcgtgcgccgtggcggcgctcaaggccgttggggaaacaacaatcgaacatgccgaagcggttaacaagtcttaccctgatttctactcagatttgaagcagctcgggggcgtggtgtctctgaaccatcagttcaacttctcttag

SEQ ID NO: 5 - protein, 414 aa, Sulfolobus solfataricus
MIVKIYPSKISGIIKAPQSKSLAIRLIFLSLFTRVYLHNLVLSEDVIDAIKSVRALGVKVKNNSEFIPPEKLEIKERFIKLKGSATTLRMLIPILAAIGGEVTIDADESLRRRPLNRIVQALSNYGISFSSYSLPLTITGKLSSNEIKISGDESSQYISGLIYALHILNGGSIEILPPISSKSYILLTIDLFKRFGSDVKFYGSKIHVNPNNLVEFQGEVAGDYGLASFYALSALVSGGGITITNLWEPKEYFGDHSIVKIFSEMGASSEYKDGRWFVKAKDKYSPIKIDIDDAPDLAMTIAGLSAIAEGTSEIIGIERLRIKESDRIESIRKILGLYGVGSEVKYNSILIFGINKGMLNSPVTDCLNDHRVAMMSSALALVNGGVITSAECVGKSNPNYWQDLLSLNAKISIE

SEQ ID NO: 6 - protein, 424 aa, Fusobacterium nucleatum
MRNMNKKIIKADKLVGEVTPPPSKSVLHRYIIASSLAKGISKIENISYSDDIIATIEAMKKLGANIEKKDNYLLIDGSKTFDKEYLNNDSEIDCNESGSTLRFLFPLSIVKENKILFKGKGKLFKRPLSPYFENFDKYQIKCSSINENKILLDGELKSGVYEIDGNISSQFITGLLFSLPLLNGNSKIIIKGKLESSSYIDITLDCLNKFGINIINNSYKEFIIEGNQTYKSGNYQVEADYSQVAFFLVANSIGSNIKINGLNVNSLQGDKKIIDFISEIDNWTKNEKLILDGSETPDIIPILSLKACISKKEIEIVNIARLRIKESDRLSATVQELSKLGFDLIEKEDSILINSRKNFNEISNNSPISLSSHSDHRIAMTVAIASTCYEGEIILDNLDCVKKSYPNFWEVFLSLGGKIYEYLG

SEQ ID NO: 7 - protein, 17 aa, Artificial Sequence
  Consensus translation
  VARIANT 2: Xaa = Asp, Lys, Glu or Asn
  VARIANT 3: Xaa = Cys, Leu, Phe or Trp
  VARIANT 4: Xaa = Gly, Asn, Arg, Glu, Lys or Ser
  VARIANT 5: Xaa = Glu or Gly
  VARIANT 7: Xaa = Gly or Asp
  VARIANT 8: Xaa = Leu, Ser, Arg, Ile, Thr, Met or Pro
  VARIANT 9: Xaa = Ser or Thr
  VARIANT 10: Xaa = Ile, Leu, Phe or Met
  VARIANT 12: Xaa = Met, Phe, Ile or Leu
  VARIANT 13: Xaa = Phe or Leu
  VARIANT 14: Xaa = Thr, Val, Ile or Ala
  VARIANT 16: Xaa = Ile, Leu, Phe or Met
IXXXXSXXXXRXXXPXL

SEQ ID NO: 8 - protein, 3 aa, Artificial Sequence
  Hypothetical consensus sequence
  VARIANT 2: Xaa = Arg, Cys or Trp
  VARIANT 3: Gly or Val
AXX

SEQ ID NO: 9 - protein, 3 aa, Artificial Sequence
  Hypothetical sequence #1
ARG

SEQ ID NO: 10 - protein, 3 aa, Artificial Sequence
  Hypothetical sequence #2
ARV

SEQ ID NO: 11 - protein, 3 aa, Artificial Sequence
  Hypothetical sequence #3
ACG

SEQ ID NO: 12 - protein, 3 aa, Artificial Sequence
  Hypothetical sequence #4
ACV

SEQ ID NO: 13 - protein, 3 aa, Artificial Sequence
  Hypothetical sequence #5
AWG

SEQ ID NO: 14 - protein, 3 aa, Artificial Sequence
  Hypothetical sequence #6
AWV

SEQ ID NO: 15 - DNA, 48 bp, Artificial Sequence
  consensus sequence
  misc_feature 4, 10, 11, 14, 40: r = A or G
  misc_feature 6, 8: k = G or T
  misc_feature 9, 12, 24, 30, 36, 39, 48: s = G or C
  misc_feature 22, 42: m = A or C
  misc_feature 23: b = G, C or T
  misc_feature 25, 28, 34, 46: w = A or T
  misc_feature 27, 41: y = T or C
atcraktksrrsgratcagcgmbswcywtscgcwtsttsrymccawts

SEQ ID NO: 16 - protein, 23 aa, Artificial Sequence
  Clone EVO1(2-5)
PFIDCGESGLSMRLFTPFVALSK

SEQ ID NO: 17 - protein, 23 aa, Artificial Sequence
  Clone L2-2
PFIDCDESGLSIRMFTPIVALSK

SEQ ID NO: 18 - protein, 23 aa, Artificial Sequence
  Clone L2-3
PFIDCDESGLSIRMFTPIVALSK

SEQ ID NO: 19 - protein, 23 aa, Artificial Sequence
  Clone L2-4
PFIKCRESGLSMRMFAPMVALSK

SEQ ID NO: 20 - protein, 23 aa, Artificial Sequence
  Clone L2-6
PFIDCGESGLSFRMFVPIVALSK

SEQ ID NO: 21 - protein, 23 aa, Artificial Sequence
  Clone L2-7
PFIECGESGLSIRLFTPLVALSK

SEQ ID NO: 22 - protein, 23 aa, Artificial Sequence
  Clone L2-8
PFIDCSESGLSFRMFAPLVALSK

SEQ ID NO: 23 - protein, 23 aa, Artificial Sequence
  Clone L2-9
PFINCGESGLSFRMFIPMVALSK

SEQ ID NO: 24 - protein, 23 aa, Artificial Sequence
  Clone L2-A
PFINCDESGLSFRMFTPIVALSK

SEQ ID NO: 25 - DNA, 48 bp, Artificial Sequence
  Coding sequence for GRG20 Q-loop region
atcaagttgaagggatcagcgacctctatccgcatgttcatcccaatc

SEQ ID NO: 26 - DNA, 48 bp, Artificial Sequence
  Coding sequence for GRG21 Q-loop region
atcgattgcaacgaatcagggagcaccttgcgcttcttggtcccattg